(19) United States
(12) Patent Application Publication
(10) Pub. No.: US 2026/0199229 A1
(43) Pub. Date: Jul. 16, 2026

(54) MULTILAYER LAYER ADHESIVE ORALLY DISSOLVING FILMS

(71) Applicant: PHARMEDICA LTD., Haifa (IL)

(72) Inventors: Yehoshua SHEINMAN, Mizpe Ilan (IL); Odeya BEN YAAKOV TAIRY, Hashmonaim (IL); Hadas GOLDBERG, Rehovot (IL); Yoram RUBIN, Ramat Hasharon (IL)

(73) Assignee: PHARMEDICA LTD., Haifa (IL)

(21) Appl. No.: 19/024,087

(22) Filed: Jan. 16, 2025

(51) Int. Cl. A61K 9/00; A61K 31/045; A61K 47/10; A61K 47/20; A61K 47/22; A61K 47/32; A61K 47/34; A61K 47/38

(52) U.S. Cl. CPC A61K 9/006; A61K 31/045; A61K 47/10; A61K 47/20; A61K 47/22; A61K 47/32; A61K 47/34; A61K 47/38

(57) ABSTRACT

An orally dissolving film (ODF) is adapted to adhere to an oral mucosal tissue or gum tissue of a subject and a method for preparing the ODF. The ODF contains wells that can be filled with one or more pharmaceutical agents. The agents can be filled during the ODF production or before administration to allow personal dosing and agent combinations.

### FIELD OF THE INVENTION

The invention relates to orally dissolving film formulations that adhere to the oral mucosal tissue or the gum tissue and deliver active ingredients in a trans-buccal and gum tissue manner and/or to the mouth cavity.

### BACKGROUND OF THE INVENTION

Orally dissolving films (ODF) were previously developed to release an active ingredient in a sustained and controlled manner.

Some ODFs focused on administration of agents efficient in managing various oral conditions, including halitosis, dry mouth, as well as various conditions associated with dentures.

In other cases, polymer compositions for drug delivery were developed. These ODFs were designed to adhere to the oral mucosal tissue and deliver an active ingredient to the mouth cavity. The active ingredient is then released in a sustained and controlled manner, affecting the oral salivary glands.

Such ODF formulations were based on a unique combination of polymers characterized in that they interact with each other in a manner that inhibits dissolution. The strong interaction between the polymers results with a high viscosity suspension due to increased entanglement of adjacent polymeric segments. This results in slow dissolvable ODFs.

Typical ODF formulations of this nature include a Carbomer and/or other mucoadhesive polymers. Such ODFs limit the amount of API available in the formulation and the duration of the sustained release formulation.

A drawback of such ODF formulations is the existence of adhesion problems in subjects without dry mouth (i.e. with normal saliva conditions). This limits the use of ODFs in people without dry mouth conditions.

An additional drawback of these formulations is the need to keep the main ingredients of the ODF in a narrow band ratio. This may lead to a reduced active volume of the active pharmaceutical agent and may limit the total amount of the active pharmaceutical agent. Further, the formulation preparation process can impair the stability and efficacy of the active pharmaceutical agent. In addition, variations in the quantity of the active pharmaceutical agent in the ODF may occur, which may render the production process costly and inefficient.

In view of the disadvantages detailed above, a mucosal adhesive oral film for trans buccal or gum release of an active agent would be helpful. It is, therefore, an object of this invention to provide an ODF capable of prolonged controlled release of drugs into the oral cavity and trans-buccal administration.

It is another object of this invention to provide a film, for continual release of active agents, that has better adherence to the mucosa and is suitable for use in subjects without dry mouth, i.e., under normal saliva conditions.

It is yet another object of this invention to provide an adhesive film for placing on the mouth mucosa or gum tissue, which has a higher percentage of active pharmaceutical ingredient available for release.

A further object of this invention aims at the provision of a process for preparing ODFs with superior qualities.

This invention also aims at providing a film for effectively treating or preventing conditions that require systemic administration of APIs and treating or preventing mouth conditions.

### SUMMARY OF THE INVENTION

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, and further comprising Hydroxypropyl methylcellulose (HPMC) powder with an average particle size of 50 to 150 μm on a surface of one active layer of said at least one active layers for attaching to the buccal mucosa tissue or the gum tissue.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, and wherein the second layer is a second active layer.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the second layer is a second active layer, and wherein the second active layer is adhered to at least one additional layer second active layer is adhered to at least one additional layer.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the wherein the second layer is a backing layer.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the wherein the second layer is a backing layer, and wherein the backing layer comprises hydrophobic polymers or other polymers that dissolve slower than the first active layer.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the first active layer and the second layer are made of thermoplastic material.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent has a molecular mass below 300 Daltons.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent is one active pharmaceutical agent with different release profiles.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent is released by controlled release from the surface of the first active layer when attached to the buccal mucosa within three hours.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, for use in treating or preventing a systemic condition in a patient with normal saliva conditions.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, further having an active pharmaceutical ingredient mixed into the first active layer and/or a second layer.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein a portion of the plurality of wells are filled with one or more active pharmaceutical agents during ODF production.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent is added prior to patient administration, and wherein the amount of the at least one active pharmaceutical agent added prior to patient administration is personalized for treatment of a patent to be administered the ODF.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein a portion of the plurality of wells are filled with one or more active pharmaceutical agents during ODF production, and wherein another portion of the plurality of wells are filled with an amount of the at least one active pharmaceutical agent added prior to patient administration personalized for treatment of a patent to be administered the ODF.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein each layer of the ODF has a thickness of up to 1 mm.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the ODF has an area of 2-4 cm².

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the ODF is oval in shape having a radius of 5-20 mm.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the wells have a diameter of 0.5-6 mm.

According to an embodiment of the invention, there is provided an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein each well has a volume of 0.5-23 mm³.

According to an embodiment of the invention, there is provided a method for delivering a systemic active pharmaceutical agent to a patient, comprising applying to a mucosal tissue or gum tissue an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent.

According to an embodiment of the invention, there is provided a method for delivering a systemic active pharmaceutical agent to a patient, comprising applying to a mucosal tissue or gum tissue an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein said mucosal tissue is the buccal mucosa, palate mucosa, sublingual mucosa, or gingival mucosa.

According to an embodiment of the invention, there is provided a method for preparing an orally dissolving film (ODF) for adhering to and administering an active pharmaceutical agent to the buccal mucosa or a gum tissue of a subject, said method comprising: providing a first active layer for attaching to a buccal mucosa or the gum tissue of the subject; punching in said first active layer a plurality of holes; providing a second layer to the first active layer, serving as a barrier for the holes, so as to form a plurality of wells; adhering the first layer to the second layer; and dispensing the active pharmaceutical agent adhering the first layer to the second layer.

According to an embodiment of the invention, there is provided a method for preparing an orally dissolving film (ODF) for adhering to and administering an active pharmaceutical agent to the buccal mucosa or a gum tissue of a subject, said method comprising one or more of the following steps: i) a first active layer is extruded from an HME extruder with a temperature of 80 C on to a cooling conveyor, ii) well holes are punched into the first active layer, iii) the second layer is extruded from an HME extruder with a temperature of 80 C on to a cooling conveyor, iv) the first active layer and second layer are bound together by hot press rollers, v) the wells are cleaned and then the drug material is micro-dispensed into the wells as the combined layers are moved on a conveyor, vi) a powder applicator applies the fine HPMC powder to one surface of the first active layer, and vii) suction unit collects excess HPMC not applied to the first active layer.

According to an embodiment of the invention, there is provided a method for preparing an orally dissolving film (ODF) for adhering to and administering an active pharmaceutical agent to the buccal mucosa or a gum tissue of a subject, said method comprising one or more of the following steps: i) a first active layer prepared by solvent casting a roll is placed on a conveyor, ii) well holes are punched into the first active layer, iii) the second layer is prepared by solvent casting into a roll and said roll is placed on conveyor, iv) the first active layer and second layer are bound together by hot press rollers, v) the wells are cleaned and then the drug material is micro-dispensed into the wells as the combined layers are moved on a conveyor, vi) a powder applicator applies the fine HPMC powder to one surface of the first active layer, and vii) suction unit collects excess HPMC not applied to the first active layer

### BRIEF DESCRIPTION OF THE DRAWINGS

In order for the present invention, to be better understood and for its practical applications to be appreciated, the following Figures are provided and referenced hereafter. It should be noted that the Figures are given as examples only and in no way limit the scope of the invention.

FIG. 1 illustrates a schematic view of the thermoplastic and mechanical binding process.

FIG. 2 illustrates a schematic view of the fine HPMC powder on the portion of the active layer for attachment to the buccal tissue surface and the buccal tissue surface to which the portion of the active layer will be attached.

FIG. 3 illustrates a schematic view of the application of hydrophilic powder on the active layer.

FIG. 4 illustrates a schematic view of the active layer of the film with wells and circles that can be punched to shape the layer.

FIG. 5 illustrates a schematic view of the ODF with an active layer with fine HPC powder for attachment to the buccal mucosa or gum tissue.

FIG. 6 illustrates a schematic view of the multilayer production process.

FIG. 7 illustrates another schematic view of the multilayer production process.

FIG. 8 illustrates a schematic view of a 22 mm diameter circular shape patch.

### DETAILED DESCRIPTION OF THE INVENTION

It is noted that throughout, all ranges and numeric figures are considered to be approximate even when the term “about” is not used, such that the document is considered to cover ±10% of the disclosed range or figure, unless specifically mentioned otherwise. It is further noted that the term “about” is also considered to cover ±10% of the disclosed range or figure, unless specifically mentioned otherwise.

It is further noted that throughout, the term “active agent” and “active ingredient” are defined to include any agent that provides a desired therapeutic effect. Thus, any natural or chemical agents that provide a desired therapeutic effect, either in the oral cavity or systemically, are included in the term “active agent” or “active ingredient”. For example, not only pharmaceutically known active agents are included; also, any other beneficial agents, and the like are included in the definition of “active agent” or “active ingredient” as used herein. The “drug”, “active pharmaceutical ingredient” or “active pharmaceutical agent” refer to pharmaceutical agents that provide a desired therapeutic effect either in the oral cavity or systemically.

The term “drug carrier material” means a mixture of mucoadhesive polymers, plasticizers, and solvents with an active pharmaceutical ingredient. The “drug carrier material” can be a “self-hardening drug carrier material” which is prepared by mixing mucoadhesive polymers, plasticizers, and solvents as a high viscosity liquid and mixing the high viscosity liquid with an active pharmaceutical agent.

It is further noted that throughout, the term “layer” relates to a film layer in the ODF. The term “backing layer” and “inactive layer” each refer to a layer that substantially blocks the drug transport to the mouth cavity and does not contain an active agent. The term “active layer” relates to a layer that contains drug content and has drug transport capabilities.

The terms “dissolvable” or “erodible” as used herein are interchangeable, unless specifically mentioned otherwise or unless a person skilled in the art would have understood those terms to have different meanings in specific instances. The terms “dissolvable” and “erodible” may be used to describe a film that dissolves in the mouth cavity, or that decomposes while its components are essentially dissolved or removed from the mouth cavity within the desired time.

The term “immediate release” or “fast release” drug profile relates to a drug profile that substantially releases the drug within 30 minutes.

The term “controlled release” drug profile relates to a drug profile that releases the drug over a period greater than 30 minutes. The term “sustained” drug profile relates to a drug profile that releases the drug over a period greater than 2 hours.

The term “personalized treatment” relates to the amount and release of the active pharmaceutical agent tailored to the treatment of a specific patient or patient group.

Some embodiments of the invention include an orally dissolving film (ODF) for adhering to and administering an active pharmaceutical agent to the buccal mucosa or the gum tissue of a subject, said ODF comprising at least one active layer for attaching to a buccal mucosa or the gum tissue of the subject.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, further comprising Hydroxypropyl methylcellulose (HPMC) powder with an average particle size of 50 to 150 μm on a surface of the first active layer for attaching to the buccal mucosa tissue or the gum tissue.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the second layer is a second active layer.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the second layer is a second active layer, and wherein the second active layer is adhered to at least one additional layer.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the second layer is a backing layer.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the second layer is a backing layer, and wherein the backing layer comprises hydrophobic polymers that dissolve slower than the first active layer.

Some embodiments of the invention include an orally dissolving film (ODF) for adhering to and administering an active pharmaceutical agent to the buccal mucosa or the gum tissue of a subject, said ODF comprising at least two active layers adhered together.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the first active layer and the second layer are made of thermoplastic material.

Some embodiments of the invention include an ODF composed of a thin film with strong thermoplastic properties to ensure strong bonding.

Some embodiments of the invention involve a method for preparing a thin film with strong thermoplastic properties by solvent casting or hot melt extrusion of the powder and/or liquid ingredients.

Some embodiments of the invention include an orally dissolving film (ODF) for adhering to and administering an active pharmaceutical agent to the buccal mucosa or the gum tissue of a subject, said ODF comprising at least two active layers adhered together, and wherein the at least two active layers are adhered together mechanically (by heat and pressure), using the thermoplastic properties of the layer materials.

Some embodiments of the invention involve a backing layer with hydrophobic polymers that dissolve slower than the active layer and thermoplastic polymers to bind the backing layer to an active layer.

Some embodiments of the invention involve a backing layer with thermoplastic and mechanical binding properties.

Some embodiments of the invention involve a backing layer and at least one active layer bound by thermoplastic and mechanical binding.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent is one active pharmaceutical agent with different release profiles.

Some embodiments of the invention include an orally dissolving film (ODF) for adhering to and administering an active pharmaceutical agent to the buccal mucosa or the gum tissue of a subject, said ODF comprising at least two active layers adhered together, wherein the at least two active layers adhered together have active pharmaceutical agents with different release profiles and/or different active pharmaceutical agents, and wherein an active layer has a surface exposed to the oral cavity.

Some embodiments of the invention include an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, wherein each active layer of said at least one active layer comprises wells containing a drug carrier material that includes an active pharmaceutical agent.

Some embodiments of the invention include a multilayer ODF with two or more layers. At least one of the layers containing a polymer composition that allows for an immediate fast release drug profile. At least one additional layer containing a polymer composition that allows for a sustained or controlled release drug profile.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent has a molecular mass below 300 Daltons.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent is released by controlled release from the surface of the first active layer when attached to the buccal mucosa within three hours.

Some embodiments of the invention include a multilayer ODF capable of drug release to both mucosal tissue or gum tissue for treatment of systemic conditions and the oral cavity for treatment of conditions in the oral cavity.

Some embodiments of the invention include a method for delivering a systemic active pharmaceutical agent to a patient, comprising applying to a mucosal tissue or gum tissue an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent.

Some embodiments of the invention include a method for delivering a systemic active pharmaceutical agent to a patient, comprising applying to a mucosal tissue or gum tissue an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein said mucosal tissue is the buccal mucosa, palate mucosa, sublingual mucosa, or gingival mucosa

Some embodiments of the invention include a multilayer ODF with unidirectional release for trans-buccal administration of at least one active pharmaceutical ingredient by using an outer layer of film, formulated to with hydrophobic polymers that dissolve slowly, and that does not contain an active pharmaceutical ingredient. The active pharmaceutical ingredient containing active layer is attached to the mucosal tissue, while the backing layer prevents drug diffusion into the mouth cavity.

Some embodiments of the invention include an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, wherein an active layer of the one or more active layers is attached to a backing layer, and wherein the backing layer comprises hydrophobic polymers that dissolve slower than the active layer.

Some embodiments of the invention include an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, wherein an active layer of the one or more active layers is attached to a backing layer, and wherein the backing layer has a different thickness than the active layer.

Some embodiments of the invention include an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, wherein an active layer of the one or more active layers is attached to a backing layer, and wherein the backing layer comprises hydrophobic polymers that dissolve slower than the active layer, and wherein two or more active layers are attached to each other and wherein each active layer has a different release profile for a pharmaceutical active agent and/or different pharmaceutical active agents, and wherein one of the two or more active layers is attached to a backing layer.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein a portion of the plurality of wells are filled with one or more active pharmaceutical agents during ODF production.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the at least one active pharmaceutical agent is added prior to patient administration, and wherein the amount of the at least one active pharmaceutical agent added prior to patient administration is personalized for treatment of a patent to be administered the ODF.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein a portion of the plurality of wells are filled with one or more active pharmaceutical agents during ODF production, and wherein another portion of the plurality of wells are filled with an amount of the at least one active pharmaceutical agent added prior to patient administration personalized for treatment of a patent to be

Some embodiments of the invention involve punching or cutting the ODF or ODF layers into geometric shapes and sizes.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the ODF has a thickness of up to 1 mm.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the ODF has an area of 2-4 cm².

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the first active layer is oval in shape having a radius of 5-20 mm.

Some embodiments of the invention involve punching multiple small holes into the ODF or active ODF film layers which serve as wells for a drug carrier material with high drug concentration.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the wells have a diameter of 0.5-6 mm.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the wells have a diameter of 0.5-6 mm, and wherein Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, wherein the wells have a diameter of 0.5-6 mm.

Some embodiments of the invention involve a layer with small holes punched into the active ODF film layer.

Some embodiments of the invention involve covering small holes punched into the active ODF film layer with HPMC fine powder, and another active layer, or a non-active backing layer.

Some embodiments of the invention involve a method for preparing an orally dissolving film (ODF) for adhering to and administering an active pharmaceutical agent to the buccal mucosa or a gum tissue of a subject, said method comprising: providing a first active layer for attaching to a buccal mucosa or the gum tissue of the subject; punching in said first active layer a plurality of holes; providing a second layer to the first active layer, serving as a barrier for the holes, so as to form a plurality of wells; adhering the first layer to the second layer; and dispensing the active pharmaceutical agent adhering the first layer to the second layer.

Some embodiments of the invention involve a method for preparing a thin film with strong thermoplastic properties by solvent casting or hot melt extrusion of the powder and/or liquid ingredients with a fine HPMC powder with an average particle size of 50 to 150 μm added to the surface of the active layer to be attached to the buccal mucosa or gum tissue.

Some embodiments of the invention involve a method for preparing a thin film with strong thermoplastic properties by solvent casting or hot melt extrusion of the powder and/or liquid ingredients with a fine HPMC powder with an average particle size of 50 to 150 μm added to the surface of the active layer for attachment to the buccal mucosa comprising one or more of the following steps: i) an active layer is extruded from an HME extruder with a temperature of 80 C on to a cooling conveyor, ii) a powder applicator applies the fine HPMC powder to one surface of the active layer, iii) axial blowers cool the active layer as the active layer moves on a cooling conveyor, and iv) a pressing roller that presses the HPMC onto the active layer.

Some embodiments of the invention involve a method for preparing a self-hardening drug carrier material comprising one or more of the following steps: i) mixing mucoadhesive polymers, plasticizers, and solvents into a high viscosity liquid, ii) mixing the high viscosity liquid with the active pharmaceutical ingredient, and iii) dispensing the drug into the wells in the active layer at a temperature of 50-80 C.

Some embodiments of the invention involve a method for preparing a self-hardening drug carrier material comprising one or more of the following steps: i) mixing Xanthan gum, Carbobol 971P, PEG400 and a mix of ethanol and acetone into a high viscosity liquid, ii) mixing the high viscosity liquid with the active pharmaceutical ingredient, and iii) dispensing the drug into the wells in the active layer at a temperature of 50-80 C.

Some embodiments of the invention involve a method for preparing a ODF with strong thermoplastic properties by hot melt extrusion of the powder and/or liquid ingredients comprising one or more of the following steps: i) an active layer is extruded from an HME extruder with a temperature of 80 C on to a cooling conveyor, ii) an inactive layer is extruded from an HME extruder with a temperature of 80 C on to a cooling conveyor, iii) the active and inactive layers are bound together by hot press rollers, and iv) punching holes for wells in the active layer and introducing the drug material into the wells;

Some embodiments of the invention involve a method for preparing an active layer of an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, comprising one or more of the following steps i) an active layer is extruded from an HME extruder with a temperature of 80 C on to a cooling conveyor, ii) well holes are punched into active layer, iii) an inactive layer is extruded from an HME extruder with a temperature of 80 C on to a cooling conveyor, iv) the active and inactive layers are bound together by hot press rollers, and v) the wells are cleaned and then the drug material is micro-dispensed into the wells as the combined layers are moved on a conveyor.

Some embodiments of the invention involve a method for preparing a ODF with strong thermoplastic properties by hot melt extrusion of the powder and/or liquid ingredients with an active layer a fine HPMC powder with an average particle size of 50 to 150 μm added to the surface of the active layer for attachment to the buccal mucosa comprising one or more of the following steps: i) an active layer is extruded from an HME extruder with a temperature of 80 C on to a cooling conveyor, ii) an inactive layer is extruded from an HME extruder with a temperature of 80 C on to a cooling conveyor, iii) the active and inactive layers are bound together by hot press rollers, iv) punching holes for wells in the active layer and introducing the drug material into the wells; v) a powder applicator applies the fine HPMC powder to one surface of the active layer, and vi) suction unit collects excess HPMC not applied to the active layer.

Some embodiments of the invention involve a method for preparing an orally dissolving film (ODF) for adhering to and administering an active pharmaceutical agent to the buccal mucosa or a gum tissue of a subject, said method comprising one or more of the following steps i) a first active layer is extruded from an HME extruder with a temperature of 80 C on to a cooling conveyor, ii) well holes are punched into the first active layer, iii) the second layer is extruded from an HME extruder with a temperature of 80 C on to a cooling conveyor, iv) the first active layer and second layer are bound together by hot press rollers, v) the wells are cleaned and then the drug material is micro-dispensed into the wells as the combined layers are moved on a conveyor, vi) a powder applicator applies the fine HPMC powder to one surface of the first active layer, and vii) suction unit collects excess HPMC not applied to the first active layer.

Some embodiments of the invention involve a method for preparing an orally dissolving film (ODF) for adhering to and administering an active pharmaceutical agent to the buccal mucosa or a gum tissue of a subject, said method comprising one or more of the following steps: i) a first active layer prepared by solvent casting a roll is placed on a conveyor, ii) well holes are punched into the first active layer, iii) the second layer is prepared by solvent casting into a roll and said roll is placed on conveyor, iv) the first active layer and second layer are bound together by hot press rollers, v) the wells are cleaned and then the drug material is micro-dispensed into the wells as the combined layers are moved on a conveyor, vi) a powder applicator applies the fine HPMC powder to one surface of the first active layer, and vii) suction unit collects excess HPMC not applied to the first active layer.

Some embodiments of the invention involve a method for preparing an orally dissolving film (ODF) for adhering to and administering an active pharmaceutical agent to the buccal mucosa or a gum tissue of a subject, said method comprising, a first active layer prepared by solvent casting, and a second layer is prepared by HME extrusion.

Some embodiments of the invention involve a method for preparing an orally dissolving film (ODF) for adhering to and administering an active pharmaceutical agent to the buccal mucosa or a gum tissue of a subject, said method comprising, a first active layer prepared by HME extrusion, and a second layer is prepared by solvent casting.

Some embodiments of the invention involve at least one active layer designed to be attached to the buccal membrane or the gum tissue.

Some embodiments of the invention include an orally dissolving film (ODF) for adhering to and administering an active pharmaceutical agent to the buccal mucosa or a gum tissue of a subject, said ODF comprising: a first active layer for attaching to a buccal mucosa or the gum tissue of the subject, said first active layer comprising a plurality of holes; and a second layer adhered to the first active layer, serving as a barrier for the holes, so as to form a plurality of wells containing at least one active pharmaceutical agent, for use in treating or preventing a systemic condition in a patient with normal saliva conditions.

Some embodiments of the invention involve at least one active layer designed to be attached to the buccal membrane or gum tissue capable of attachment to the buccal membrane or gum tissue with normal to high moisture.

Some embodiments of the invention involve at least one active layer designed to be attached to the buccal membrane or gum tissue capable of attachment to the buccal membrane or gum tissue in patients without dry mouth, i.e. with normal saliva conditions.

Some embodiments of the invention involve at least one active layer designed to be attached to the buccal membrane or gum membrane capable of attachment to buccal membrane or gum tissue with normal to high moisture and dry mouths, i.e., with normal saliva conditions.

Some embodiments of the invention involve at least one active layer designed to be attached to the buccal membrane with fine Hydroxypropyl methylcellulose (HPMC) powder with an average particle size of 50 to 150 μm on the side of the surface of the active layer facing the buccal tissue for use in patients without dry mouth, i.e. with normal saliva conditions.

Some embodiments of the invention involve a method for binding a backing layer and at least one active layer using a hot-pressing roller that binds a backing layer from a roll of backing layer and an active layer roll of active layer.

Some embodiments of the invention involve a backing layer that contains a coloring agent to distinguish the backing layer from active layers.

Some embodiments of the invention involve a backing layer adapted to adhere to an active layer, wherein the backing layer comprises one or more of i) at least one mucoadhesive constituting from 23% to 90% of the backing layer's dry weight; ii) at least one plasticizer constituting from 10% to 60% of the backing layer's dry weight; iii) optionally at least one breath freshener ingredient constituting up to 5% of the backing layer's dry weight; iv) optionally at least one sweetener agent constituting up to 5% of the backing layer's dry weight; and v) optionally at least one coloring agent constituting up to 0.1% of the backing layer's dry weight.

Some embodiments of the invention involve a backing layer adapted to adhere to an active layer, wherein the backing layer comprises one or more of i) hydroxypropyl cellulose from 10% to 70% of the backing layer's dry weight; ii) Xanthan gum from 3-30% of the backing layer's dry weight; iii) Carbopol 971P from 10-50% of the backing layer's dry weight; iv) PEG400 from 5-30% of the backing layer's dry weight; v) Propylene glycol from 5-30% of the backing layer's dry weight; vi) optionally menthol constituting up to 5% of the backing layer's dry weight; vii) optionally Xylitol constituting up to 5% of the backing layer's dry weight; viii) optionally Saccharin constituting up to 5% of the backing layer's dry weight; and ix) optionally Allura red constituting up to 0.1% of the backing layer's dry weight.

Some embodiments of the invention involve an active layer adapted to adhere to at least one additional layer and optionally the buccal mucosa or gum tissue, wherein the active layer contains wells containing a drug carrier material comprising one or more of i) at least one mucoadhesive constituting from 10% to 25% of the active layer's dry weight; ii) at least one plasticizer constituting from 10% to 25% of the active layer's dry weight; iii) at least one solvent ingredient constituting from 10% to 20% of the active layer's dry weight; and iv) at least one active pharmaceutical agent constituting from 30-90% of the active layer's dry weight.

Some embodiments of the invention involve the active layer adapted to adhere to at least one additional layer and optionally the buccal mucosa or gum tissue, wherein the active layer contains wells containing drug carrier material comprising one or more of i) Xanthan gum from 5% to 10% of the active layer's dry weight; ii) Carbopol 971P from 3-15% of the active layer's dry weight; iii) PEG400 from 10-25% of the active layer's dry weight; iv) a mix of ethanol and acetone from 5-10% of the backing layer's dry weight; and v) at least one active pharmaceutical agent constituting from 30-90% of the active layer's dry weight.

Some embodiments of the invention involve an active agent released from the surface of the active layer attached to the buccal mucosa or gum tissue within eight hours or more for treating or preventing a systemic condition, constituting from 30% to 90% of the active layer dry weight; wherein a fine HPMC powder with an average particle size of 50 to 150 μm is added to the surface of the active layer attached to the buccal mucosa.

Some embodiments of the invention comprise an active agent released from the surface of the active layer attached to the buccal mucosa or gum tissue within eight hours or more for treating or preventing a systemic condition in a patient without dry mouth (i.e. with normal saliva conditions), constituting from 30% to 90% of the active layer dry weight; wherein a fine HPMC powder with an average particle size of 50 to 150 μm is added to the surface of the active layer attached to the buccal mucosa.

Some embodiments of the invention comprise an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, and wherein an active layer of said at least one active layer includes an active pharmaceutical agent to be released by controlled release from the surface of that active layer when attached to the buccal mucosa within three hours.

Some embodiments of the invention comprise an orally dissolving film (ODF) for adhering to and administering an active pharmaceutical agent to the buccal mucosa or the gum tissue of a subject, said ODF comprising: at least one active layer for attaching to a buccal mucosa or the gum tissue of the subject, wherein an active layer of said at least one active layer includes an active pharmaceutical agent to be released by controlled release from the surface of that active layer when attached to the buccal mucosa within three hours, and wherein active pharmaceutical agent constitutes from 30% to 90% of the active layer dry weight.

Some embodiments of the invention comprise active agent released from the surface of the active layer attached to the buccal mucosa or gum tissue within three hours or more for treating or preventing a systemic condition in a patient with normal saliva conditions, constituting from 30% to 90% of the active layer dry weight; wherein a fine HPMC powder with an average particle size of 50 to 150 μm is added to the surface of the active layer attached to the buccal mucosa.

Some embodiments of the invention involve the attachment of multiple active layers with different release profiles and/or different active agents attached to each other, wherein fine HPMC powder with an average particle size of 50 to 150 μm is added to the surface of the active layer for attachment to the buccal mucosa.

Some embodiments of the invention involve the attachment of multiple active layers with different release profiles and/or different active agents are attached to each other, wherein fine HPMC powder with an average particle size of 50 to 150 μm is added to the surface of the active layer attached to the buccal mucosa, and wherein one of the active layers is attached to a backing layer.

Some embodiments of the invention involve the attachment of multiple active layers with different release profiles and/or different active agents attached to each other, wherein fine HPMC powder with an average particle size of 50 to 150 μm is added to the surface of the active layer attached to the buccal mucosa, and wherein a different active layer has a surface exposed to the oral cavity.

The ODF may be advantageously employed for managing oral and systemic conditions, for example oral conditions selected from the group consisting of herpetic gingivostomatitis, aphthous stomatitis, irritation fibroma, swollen floor of mouth, enlargement of tongue, glossitis, oral erythema, salivary glands swelling, dry mouth, xerostomia, gingivitis, denture sores, sore throat, and many other conditions that may benefit from controlled release of an agent in the mouth cavity.

Further embodiments of the invention are directed to a method for treating or preventing an oral condition, wherein the method comprises administering the dissolvable film of the invention and wherein the oral condition is selected from the group consisting of herpetic gingivostomatitis, aphthous stomatitis, irritation fibroma, swollen floor of mouth, enlargement of tongue, glossitis, oral erythema, salivary glands swelling, dry mouth, xerostomia, halitosis, gingivitis, denture sores, sore throat.

The ODF may be advantageously employed for managing oral and systemic conditions, for example a systemic condition selected from the group consisting of autoimmune disorders, endocrine disorders, cardiovascular conditions, infectious diseases, renal disorders, gastrointestinal disorders, neurological conditions, hematological disorders, metabolic disorders, psychiatric conditions, and musculoskeletal conditions and many other conditions that may benefit from an agent released into the buccal mucosa.

According to some embodiments, the systemic active agents are selected from systemic active agents with a molecular mass below 300 Daltons or any combination of agents below 300 Daltons.

According to some embodiments, the systemic active agents are selected from the group consisting of corticosteroids, immunosuppressants, biological agents, hypoglycemic agents, thyroid agents, antihypertensives, statins, antiplatelet drugs, diuretics, antibiotics, antiviral drugs, antifungal drugs, proton pump inhibitors, laxatives, antiepileptic drugs, immunomodulators, antidepressants, antipsychotics, anxiolytics, stimulants, iron supplements, growth hormone, cholesterol lowering drugs, and bisphosphonates or any combination thereof.

The film of the invention may comprise additional active agents, such as agents being selected from the group consisting of additional breath fresheners, anti-cavity compounds, anti-anxiety agents, anti-inflammatory agents, analgesics, antihistamines, local anesthetics, anti-bacterial compounds, disinfectants, antiseptics, antibiotics, anti-fungal, anti-migraine, anti-asthmatics, cold remedies, cough remedies, nicotine, proton pump inhibitors, H₂ receptor antagonists, vitamins and other dietary and nutritional supplements.

Further embodiments of the invention are directed to a method for delivering a systemic active pharmaceutic agent to a patient, comprising applying the ODF of the invention, as detailed herein, to a mucosal tissue or gum tissue. According to some embodiments, the mucosal tissue comprises buccal mucosa, palate mucosa, sublingual mucosa, or gingival mucosa.

According to some embodiments of the invention are an ODF can be include at least one active layer with wells containing 30-90% of an active pharmaceutical ingredient, wherein the surface of the active layer is covered with fine powder HPMC with an average particle size of 50 to 150 μm to ensure a firm attachment to the buccal mucosa or gum tissue.

In some embodiments, an ODF further comprises a backing layer facing the oral cavity to ensure that the ODF substantially does not release active pharmaceutical ingredient into the oral cavity.

In some embodiments, an ODF comprises multiple active layers with different release profiles, such as immediate and controlled release.

In some embodiments, an ODF comprises multiple active layers with one active layer surface covered with fine powder HPMC with an average particle size of 50 to 150 μm facing the buccal mucosa or gum tissue and another active layer surface facing the oral cavity.

According to some embodiments, an ODF may be implemented and replaced with another ODF by the patient as desired. According to some embodiments, an ODFs may be implemented/replaced in any desired frequency, e.g., once an hour, once a day, once every eight hours, once every twelve hours, once a week, once every other day, and the like.

The ODF layers of the invention may exhibit a smooth surface.

According to some embodiments, the ODF may have a thickness of 0.2-1.0 mm. According to some embodiments, the thickness is up to 1 mm, such as up to 0.8 mm, for example up to 0.7 mm, for example up to 0.5 mm, for example up to 0.4 mm, for example up to 0.3 mm, for example up to 0.25 mm, for example up to 0.2 mm.

According to some embodiments, the active or inactive layer may have a thickness of up to 0.8 mm. According to some embodiments, the ODF has a thickness of up to 0.5 mm, such as up to 0.45 mm, for example up to 0.40 mm, for example up to 0.3 mm, for example up to 0.25 mm, for example up to 0.24 mm, for example up to 0.23 mm, for example up to 0.22 mm, for example up to 0.2 mm.

According to some embodiments, the active or inactive layer may have an area of 2-4 cm². According to some embodiments, the ODF has an area of up to 4 cm², such as up to 3.5 cm², for example up to 3 cm², for example up to 2.5 cm², for example up to 2 cm².

According to some embodiments, the active or inactive layer may be circular in shape having a radius of 5-20 mm. According to some embodiments, the layer has a radius of up to 20 mm, such as up to 15 mm, for example up to 13 mm, for example up to 11 mm, for example up to 10 mm, for example up to 7 mm, for example up to 5 mm.

According to some embodiments, the active or inactive layer may be rectangular in shape having an area of 1-4 cm². According to some embodiments, the layer has an area of up to 4 cm², such as up to 3.5 cm², for example up to 3 cm², for example up to 2.5 cm², for example up to 2 cm², for example up to 1.5 cm², for example up to 1 cm².

According to some embodiments, the ODF may be configured to enable the release of an active agent into the oral mucosa or to the mouth. The ODF may be in designed in any shape or form (such as square, rectangular, circular, oval, etc.). According to some embodiments, the thickness of the ODF is between about 0.2-1.0 mm.

According to some embodiments, an ODF area (e.g., length×width) may be chosen to be in the range of 5 cm² to 9 cm². According to some embodiments, an ODF may be cut to any shape and/or size. According to some embodiments, the active film of the ODF may contain wells. In some embodiments such wells may be provided with a volume of 0.5-23 mm³, well diameters of 0.5-6 mm, and a total of number of wells ranging between 5-30.

According to some embodiments, the wells in the active layers may have a diameter of 0.5-6 mm. According to some embodiments, the diameter is up to 6 mm, such as up to 5 mm, for example up to 4 mm, for example up to 3 mm, for example up to 2 mm, for example up to 1 mm, for example up to 0.5 mm.

According to some embodiments, the wells in the active layers may have a volume of 0.5-23 mm³. According to some embodiments, the volume is up to 20 mm³, such as up to 15 mm³, for example up to 10 mm³, for example up to 5 mm³, for example up to 3 mm³, for example up to 1 mm³, for example up to 0.5 mm³.

According to some embodiments, each active layer has 5-30 wells. According to some embodiments, the number of wells is up to 30, such as up to 25, for example up to 20, for example up to 15, for example up to 10, for example up to 8, for example up to 5.

According to some embodiments, each well in the active layer is filed with drug at 30-90% of the well's volume. According to some embodiments, the drug volume is up to 90%, such as up to 70%, for example up to 60%, for example up to 50%, for example up to 40%, for example up to 30%.

Some embodiments of the invention are directed to a method for preparing a layered ODF adhering to a mouth mucosa, and for controlled release within three hours or more a therapeutic or other agent to the mouth cavity and/or to the buccal mucosa/gum tissue, the film not feeling unpleasant in mouth and being dissolved within the three hours or more.

Some embodiments of the invention are directed to a method for preparing a layered ODF adhering to a mouth mucosa, and for releasing within 8 hours or more a therapeutic or other agent to the mouth cavity and/or to the buccal mucosa/gum tissue, the film not feeling unpleasant in mouth and being dissolved within 8 hours or more.

According to some embodiments, the film of the invention further comprises plasticizers in an amount of from 10% to 25% of the film dry weight. According to some embodiments, the film of the invention further comprises an additive in an amount of up to 4% of the film dry weight, the additive being selected from the group consisting of additional active agents, polymers adjusting the hydrophobicity of the polymer structure of said film, permeation enhancers, sweeteners, taste-masking agents, taste or flavor modifiers, plasticizers, release modifiers, pH-adjusting agents or buffering agents, preservatives or stabilizers, complexing agents, coloring agents, emulsifying agents, solvents selected from water, acetone, a alcohols, and inert fillers. According to some embodiments, the film of the invention further comprises a polyacrylic acid based polymer.

According to some embodiments, plasticizers may be included in an ODF that include propylene glycol, glycerin, PEG-4000, PEG-400, and the like. Plasticizers may have surfactant properties, and they may act as release modifiers, e.g. non-ionic detergents such as Brij 35 (polyoxyethylene (35) lauryl ether), Brij 58 (polyoxyethylene (20) acetyl ether), and the like. Plasticizers may impart flexibility to the dosage forms, and can affect the release profile of the active agent(s) therein. Polyamide (PA) based polymers suitable for admixing into the film of the invention include, for example, acrylic acid copolymers, methacrylic acid polymers, methacrylic acid copolymers, cross-linked polyacrylic acid, such as Carbopol, which is PA crosslinked with polyalkenyl ethers or divinyl glycol, examples of such Carbopols being Carbopol 971, Carbopol 974. and Carbopol 1342.

In some embodiments, an ODF prepared in accordance with the invention may be configured to be stable at 4° C., or even at room temperature. In situ, the ODF may be configured to maintain its integrity at least partially, which may be demonstrated in vitro, while imitating the human saliva solution and exposing the film for several hours, up to 8 h.

Reference is now made to the Figures.

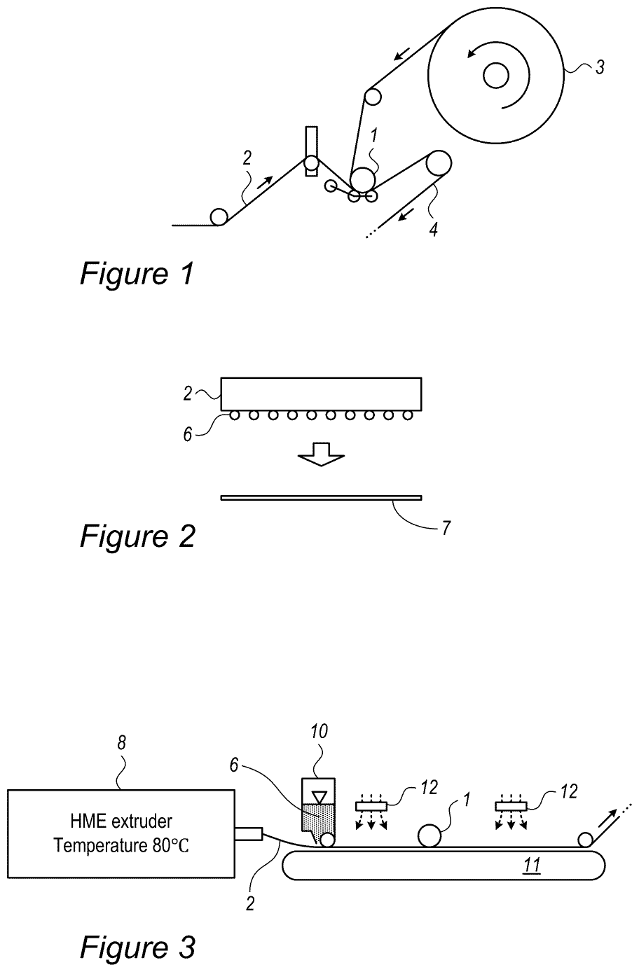

FIG. 1 illustrates a schematic view of the thermoplastic and mechanical binding process. Active layer 2 is conveyed from a roller and inactive (backing) layer 3 is conveyed from a second roller. The two layers are bound by thermoplastic and mechanical binding by a hot-pressing roller 1 and the bonded film 4 exits the roller at the opposite end.

FIG. 2 illustrates a schematic view of the fine HPMC powder 6 on the portion of the active layer for attachment to the buccal tissue surface or tissue surface and the buccal tissue surface or gum tissue surface 7 to which the portion of the active layer 2 will be attached.

FIG. 3 illustrates a schematic view of the application of hydrophilic powder on the active layer. First, an active layer 2 is extruded from an HME extruder 8 with a temperature of 80 C on to a cooling conveyor 11; next a powder applicator 10 applies the fine HPMC powder 6 to one surface of the active layer 2 the temperature at powder application is 75-80 C; next axial blower 12 is used to cool the active layer as the active layer moves on a cooling conveyor 11; finally a pressing roller 1 presses the HPMC onto the active layer.

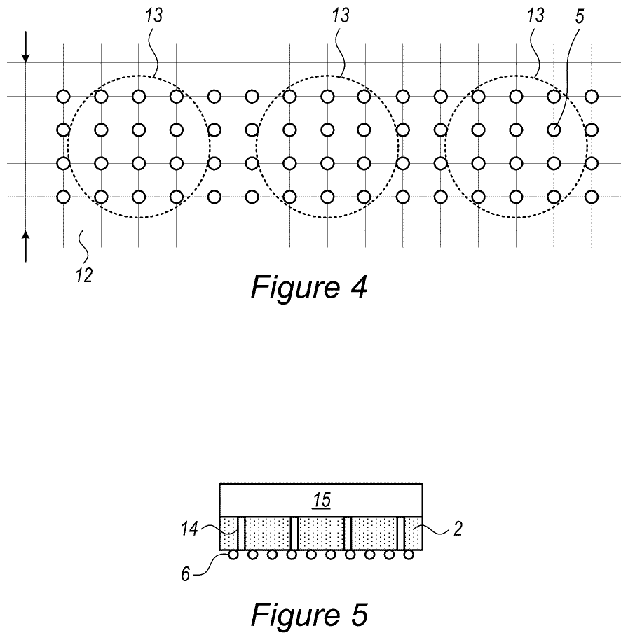

FIG. 4 illustrates a schematic view of the active layer of the film 12 with wells 5 and disks 13 that can be punched to shape the layer.

FIG. 5 illustrates a schematic view of the ODF with an active layer 2 with fine HPC powder 6 for attachment to the buccal mucosa. The active layer 2 is attached to a second layer 15. The second layer can be active or non-active layer. The active layer has holes 14 punched for wells which contain at least one active pharmaceutical ingredient.

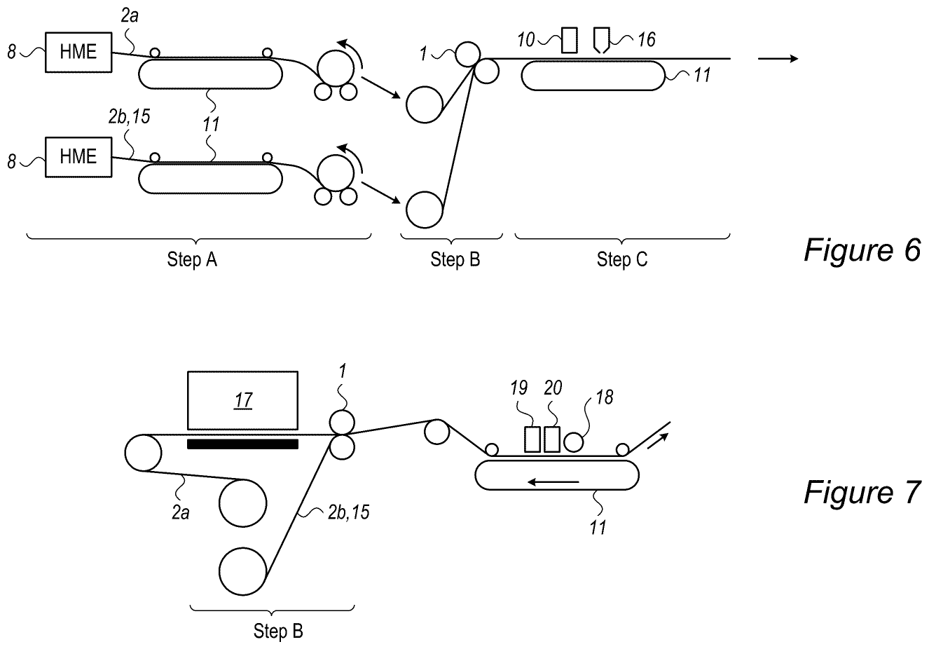

FIG. 6 illustrates a schematic view of the multilayer production process. The process involves the following steps: i) an active layer 2a is extruded from an HME extruder 8 with a temperature of 80 C on to a cooling conveyor 11, ii) a second active layer 2b or an inactive layer 15 is extruded from an HME extruder 8 with a temperature of 80 C on to a cooling conveyor 11, iii) the active layer 2a and second active layer 2b or inactive layer 15 are bound together by hot press rollers 1, iv) a powder applicator 10 applies the fine HPMC powder 6 to one surface of the active layer 2a, v) a suction unit 16 collects the excess HPMC powder 6 not affixed to the active layer 2a (excess powder is not shown); vi) the patch is formed.

FIG. 7 illustrates a schematic view of the multilayer production process. The process involves the following steps: i) a roll of active material 2a is conveyed to a punching station for punching well holes 17 and then conveyed to the hot press rollers 1, ii) a second active layer 2b or an inactive layer 15 is conveyed to the hot press rollers 1, iii) the active layer 2a and a second active layer 2b or an inactive layer 15 are bound together by hot press rollers 1, iv) wells are cleaned in area 19 on a conveyer 11 and the drug and drug carrier are dispensed in area 20 into the wells; v) a pressure roller 18 flattens the filled surface of the wells, vi) the film is the conveyed to the powder applicator 10 and suction unit 16 as in FIG. 6 (not shown in this figure).

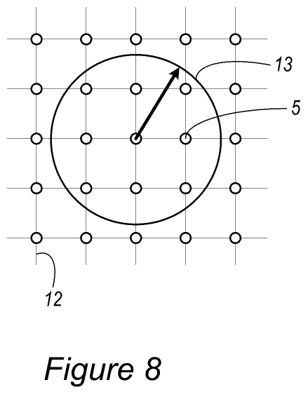

FIG. 8 illustrates a schematic view of an example of ODF having a circular/disk shape with 22 mm diameter. 13. The disk shaped patch 13 is cut from a 30 mm by 30 mm film 12. The number of wells 5 in the specific disk shaped patch 13 is nine, and the diameter of each well 5 is about 6 mm. The active layer thickness or well depth is about 0.5 mm the volume of each well is V=π×0.6²/4×0.05=0.014 cm³. The total volume of the wells in disk 13 is 0.127 cm³, this assumes that the drug density is approximately 1 gr/cm³, this disk shaped patch 13 with 50% drug volume in each well contains about 63 mg of drug.

The following examples are set forth to further describe ODFs, according to some embodiments of invention. The below examples, however, should not be construed as limiting the present invention in any manner.

### EXAMPLES

### Example 1

A backing layer of an ODF was prepared by combining components described in Table 1.

| Ingredient | | % |
|---|---|---|
| Hydroxypropyl | Mucoadhesive | 10-70 |
| cellulose | | |
| Xanthan gum | Mucoadhesive | 3-30 |
| Carbopol 971P | Mucoadhesive | 10-50 |
| PEG400 | Plasticizer | 5-30 |
| Propylene glycol | Plasticizer | 5-30 |
| Menthol | Fresh breath | 0-5 |
| Xylitol | Sweetener | 0-5 |
| Saccharin | Sweetener | 0-5 |
| Allura red | coloring agent | 0-0.1 |
| Total | | 100.0 |

### Example 2

A self-hardening drug carrier material mixed with the drug in high drug concentration for the active layer was prepared by combining components described in Table 2.

| # | Ingredient | | % |
|---|---|---|---|
| 1 | Xanthan gum | Mucoadhesive | 5-10 |
| 2 | Carbopol 971P | Mucoadhesive | 5-10 |
| 3 | PEG400 | Plasticizer | 10-25 |
| 4 | Mix Ethanol/Acetone | Solvent | 5-10 |
| 5 | API (drug) | drug | 30-50 |
| | Total | | 100% |

### Example 3

A validation test was performed with an ODF according to some embodiments of the invention to determine the attachment strength of the ODF to the buccal mucosa. Two samples were tested in groups of 17 and 30 subjects without dry mouth (i.e. with normal saliva conditions). Table 4 indicates the results of the validation test. The number of repeats indicates the number of subjects in which the ODF needed to be reattached. The “yes score” indicates the number of subjects in which the ODF had satisfactory attachment to the buccal mucosa.

| | Sample | # of | “yes” | Score | Statistical |
|---|---|---|---|---|---|
| # | size | repeats | score | in % | error (%) |
| 1 | 17 | 2 | 14 | 82 | 6.2 |
| 2 | 30 | 2 | 27.5 | 92 | 4.5 |

While the invention has been described using some specific examples, many modifications and variations are possible. It is therefore understood that the invention is not intended to be limited in any way, other than by the scope of the appended claims.

1. An orally dissolving film (ODF) for adhering to and administering an active pharmaceutical agent to the buccal mucosa or a gum tissue of a subject, said ODF comprising:
a first active layer for attaching to a buccal mucosa or the gum tissue of the subject, said first active layer comprising a plurality of holes; and
a second layer adhered to the first active layer, serving as a barrier for the holes, so as to form a plurality of wells containing at least one active pharmaceutical agent.

2. The ODF of claim 1, further comprising Hydroxypropyl methylcellulose (HPMC) powder with an average particle size of 50 to 150 μm on a surface of the first active layer for attaching to the buccal mucosa tissue or the gum tissue.

3. The ODF of claim 1, wherein the second layer is a second active layer.

4. The ODF of claim 3, wherein the second active layer is adhered to at least one additional layer.

5. The ODF of claim 1, wherein the second layer is a backing layer.

6. The ODF of claim 4, wherein the backing layer comprises hydrophobic polymers that dissolve slower than the first active layer.

7. The ODF of claim 1, wherein the first active layer and the second layer are made of thermoplastic material.

8. The ODF of claim 1, wherein the at least one active pharmaceutical agent has a molecular mass below 300 Daltons.

9. The ODF of claim 1, wherein the at least one active pharmaceutical agent is one active pharmaceutical agent with different release profiles.

10. The ODF of claim 1, wherein the at least one active pharmaceutical agent is released by controlled release from the surface of the first active layer when attached to the buccal mucosa within three hours.

11. The ODF of claim 1, for use in treating or preventing a systemic condition in a patient with normal saliva conditions.

12. The ODF of claim 1 further having an active pharmaceutical ingredient mixed into the first active layer and/or a second layer.

13. The ODF of claim 1, wherein the ODF has a thickness of up to 1 mm.

14. The ODF of claim 1, wherein the ODF has an area of 2-4 cm².

15. The ODF of claim 1, wherein the ODF is oval in shape having a radius of 5-20 mm.

16. The ODF of claim 1, wherein the wells have a diameter of 0.5-6 mm.

17. The ODF of claim 16, wherein each well has a volume of 0.5-23 mm³.

18. The ODF of claim 1, wherein a portion of the plurality of wells are filled with one or more active pharmaceutical agents during ODF production.

19. The ODF of claim 1, wherein the at least one active pharmaceutical agent is added prior to patient administration, and wherein the amount of the at least one active pharmaceutical agent added prior to patient administration is personalized for treatment of a patent to be administered the ODF.

20. The ODF of claim 1, wherein a portion of the plurality of wells are filled with one or more active pharmaceutical agents during ODF production, and wherein another portion of the plurality of wells are filled with an amount of the at least one active pharmaceutical agent added prior to patient administration personalized for treatment of a patent to be administered the ODF.

21. A method for delivering a systemic active pharmaceutical agent to a patient, comprising applying to a mucosal tissue or gum tissue an orally dissolving film (ODF) for adhering to and administering an active pharmaceutical agent to the buccal mucosa or a gum tissue of a subject, said ODF comprising:
a first active layer for attaching to a buccal mucosa or the gum tissue of the subject, said first active layer comprising a plurality of holes; and
a second layer adhered to the first active layer, serving as a barrier for the holes, so as to form a plurality of wells containing at least one active pharmaceutical agent.

22. The method for delivering a systemic active pharmaceutical agent of claim 21, wherein said mucosal tissue is the buccal mucosa, palate mucosa, sublingual mucosa, or gingival mucosa.

23. A method for preparing an orally dissolving film (ODF) for adhering to and administering an active pharmaceutical agent to the buccal mucosa or a gum tissue of a subject, said method comprising:
providing a first active layer for attaching to a buccal mucosa or the gum tissue of the subject;
punching in said first active layer a plurality of holes;
providing a second layer to the first active layer, serving as a barrier for the holes, so as to form a plurality of wells;
adhering the first layer to the second layer; and
dispensing the active pharmaceutical agent adhering the first layer to the second layer.

24. A method for preparing an orally dissolving film (ODF) for adhering to and administering an active pharmaceutical agent to the buccal mucosa or a gum tissue of a subject, said method comprising: one or more of the following steps:
i) a first active layer is extruded from an HME extruder with a temperature of 80 C on to a cooling conveyor,
ii) well holes are punched into the first active layer,
iii) the second layer is extruded from an HME extruder with a temperature of 80 C on to a cooling conveyor,
iv) the first active layer and second layer are bound together by hot press rollers,
v) the wells are cleaned and then the drug material is micro-dispensed into the wells as the combined layers are moved on a conveyor,
vi) a powder applicator applies the fine HPMC powder to one surface of the first active layer, and
vii) suction unit collects excess HPMC not applied to the first active layer.

25. A method for preparing an orally dissolving film (ODF) for adhering to and administering an active pharmaceutical agent to the buccal mucosa or a gum tissue of a subject, said method comprising: one or more of the following steps:
i) a first active layer prepared by solvent casting a roll is placed on a conveyor,
ii) well holes are punched into the first active layer,
iii) the second layer is prepared by solvent casting into a roll and said roll is placed on conveyor,
iv) the first active layer and second layer are bound together by hot press rollers,
v) the wells are cleaned and then the drug material is micro-dispensed into the wells as the combined layers are moved on a conveyor,
vi) a powder applicator applies the fine HPMC powder to one surface of the first active layer, and
vii) suction unit collects excess HPMC not applied to the first active layer.
